(19) United States
(12) Patent Application Publication
(10) Pub. No.: US 2019/0038674 A1
(43) Pub. Date: Feb. 7, 2019

(54) Formulation for the Treatment of Diabetes

(71) Applicants: Yashwant Atbhaiya, Kolkata (IN); Preetam Lal Choudhary, Raipur (IN)

(72) Inventors: Yashwant Atbhaiya, Kolkata (IN); Preetam Lal Choudhary, Raipur (IN)

(21) Appl. No.: 15/888,558

(22) Filed: Feb. 5, 2018

Related U.S. Application Data
(62) Division of application No. 14/316,308, filed on Jun. 26, 2014, now abandoned.

(30) Foreign Application Priority Data
Jun. 26, 2013 (IN) 763/KOL/2013

(51) Int. Cl. A61K 35/22; A61K 35/20

(52) U.S. Cl. CPC A61K 35/22; A61K 35/20

(57) ABSTRACT

This invention relates to a formulation for the treatment of diabetes including 56 mg to 224 mg cow urine powder and 44 mg to 176 mg skim milk powder per kg body weight of the patient.

### CROSS-REFERENCE TO RELATED APPLICATION

This application is a divisional application of U.S. patent application Ser. No. 14/316,308, filed Jun. 26, 2014, which claims priority to Indian Patent Application No. 763/KOL/2013 filed Jun. 26, 2013, the disclosure of which is hereby incorporated in its entirety by reference.

### FIELD OF THE INVENTION

This invention relates to a formulation for the treatment of diabetes.

This invention further relates to a formulation of cow urine powder and skim milk powder for the treatment of diabetes.

### BACKGROUND OF THE INVENTION

Diabetes mellitus is a serious, lifelong condition and it is a chronic disorder of carbohydrate, fat and protein metabolism characterized by increased fasting and post prandial blood sugar levels.

Diabetes mellitus is a metabolic disorder characterized by hyperglycemia and disturbances of carbohydrate, protein and fat metabolisms or relative lack of the hormone insulin. The word diabetes is derived from the “Greek” word “diabanein” which means to pass through, in reference to the excessive urine produced as a symptom of these diseases. The term diabetes, without qualification, usually refers to diabetes mellitus, which roughly translates to excessive sweet urine (known as “glycosuria”). Several rare conditions are also named diabetes. The most common of these is diabetes insipidus in which large amounts of urine are produced (polyuria), which is not sweet (insipidus meaning “without taste” in Latin). Diabetes mellitus, often simply referred to as diabetes—is a group of metabolic diseases in which a person has high blood sugar, either because the body does not produce enough insulin or because cells do not respond to the insulin that produce enough insulin or because cells do not respond to the insulin that is produced. This high blood sugar produces the classical symptoms of polyuria (frequent urination), polydipsia (increased thirst) and polyphagia (increased hunger) (www.diabetic.org.uk).

Types of diabetes:

Type I diabetes: Diabetes mellitus is characterized by loss of the insulin-producing β cells of the islets of Langerhans in the pancreas leading to insulin deficiency. This type of diabetes can be further classified as immune-mediated or idiopathic. The majority of type I diabetes is of the immune-mediated nature, where beta cell loss is a T-cell mediated autoimmune attack. There is no known preventive measure against type I diabetes, which causes approximately 10% of diabetes mellitus cases in North America and Europe. Most affected people are otherwise healthy and of a healthy weight when onset occurs. Sensitivity and responsiveness to insulin are usually normal, especially in the early stages. Type I diabetes can affect children or adults but was traditionally termed “juvenile diabetes” because it represents a majority of the diabetes cases in children.

Type II diabetes: Diabetes mellitus is characterized by insulin resistance which may” be combined with relatively reduced insulin secretion. The defective responsiveness of body tissues to insulin is believed to involve the insulin receptor. However, the specific defects are not known. Diabetes mellitus due to a known defect are classified separately. Type II diabetes is the most common type. In the early stage of type II diabetes, the predominant abnormality is reduced insulin sensitivity. At this stage hyperglycemia can be reversed by a variety of measures and medications that improve insulin sensitivity or reduce glucose production by the liver.

Cause of diabetes: Diabetes mellitus occurs when the pancreas doesn't make enough or any of the hormone insulin, or when the insulin produced does not work effectively. In diabetes, this causes the level of glucose in the blood to be too high.

In Type I diabetes, the cells in the pancreas that make insulin are destroyed, causing a severe lack of insulin. This is thought to be the result of the body attacking and destroying its own cells in the pancreas—known as an autoimmune reaction.

It's not clear why this happens, but a number of explanations and possible triggers of this reaction have been proposed. These include:

Infection with a specific virus or bacteria;

Exposure to food-borne chemical toxins.

Type II diabetes, is believed to develop when:

The receptors on cells in the body that normally respond to the action of insulin fail to be stimulated by it—this is known as insulin resistance. In response to this more insulin may be produced, and this over-production exhausts the insulin-manufacturing cells in the pancreas;

There is simply insufficient insulin available; and

The insulin that is available may be abnormal and therefore doesn't work properly.

The following risk factors increase the chances of someone developing Type II diabetes:

Increasing age

Obesity; and

Physical inactivity

Rarer causes of diabetes include:

Certain medicines

Pregnancy (gestational diabetes) and

Any illness or disease that damages the pancreas and affects its ability to produce insulin e.g. pancreatitis. (http://chinese.school.netfirms.com/diabetes.html)

Insulin is a hormone central to regulating carbohydrate and fat metabolism in the body. Insulin causes cells in the liver, muscle, and fat tissue to take up glucose from the blood, storing it as glycogen in the liver and muscle.

With the exception of the metabolic disorder diabetes mellitus and metabolic syndrome, insulin is provided within the body in a constant proportion to remove excess glucose from the blood, which otherwise would be toxic.

When control of insulin levels fails, diabetes mellitus will result. As a consequence, insulin is used medically to treat some forms of diabetes mellitus. Patients with type I diabetes depend on external insulin (most commonly injected subcutaneously) for their survival because the hormone is no longer produced internally. Patients with type II diabetes are often insulin resistant and because of such resistance, may suffer from a “relative” insulin deficiency. Some patients with type II diabetes may eventually require insulin if other medications fail to control blood glucose levels adequately. Over 40% of those with type II diabetes require insulin as part of their diabetes management plan.

A number of allopathic and hamoeopathic remedies have been tried by several workers, for reducing blood glucose levels in animal model and humans.

Adeneye and Agbaje (2007) studied hypoglycemic and hypolipidemic effects of fresh leaf aqueous extract of Cymbopogon citratus Stapf. in rats.

Yang et al. (2010) studied the anti-diabetic effect of Panax notoginseng saponins and its major anti-hyperglycemic components. Panax notoginseng (Burk) Chen F. H. (Araliaceae) is a well-known and commonly used traditional Chinese herb for treatment of various diseases, such as hemostasis, edema and odynolysis.

Ghule et al. (2009) studied the antihyperlipidemic effect of the methanolic extract from Lagenaria siceraria stand fruit in hyperlipidemic rats. Lagenaria siceraria stand (Cucurbitaceae) fruits have been used, traditionally, in the treatment of hyperlipidemia and atherosclerotic impasse and considered as cardioprotective and cardiotonic drug.

Sabu and Kuttan (2004) studied the antidiabetic activity of aegle marmelos and its relationship with its antioxidant propertyies.

Vijayakumar et al. (2005) studied the hypoglycaemic activity of fenugreek seed extract is mediated through the stimulation of an insulin signalling pathway.

However, these remedies entail the collection and use of plants, herbs, fruits, eaves etc., and preparation of extracts therefrom. Therefore, it would be advantageous to provide a remedy which is cheap, does not require the collection of herbs, fruits etc. or their processing into forms suitable for consumption.

### SUMMARY OF THE INVENTION

It is therefore an object of this invention to propose a formulation for the treatment of diabetes, which is cheap.

It is a further object of this invention to propose a formulation for the treatment of diabetes, which does not require the use of biological resources.

Another object of this invention is to propose a formulation for the treatment of diabetes, which is based on a waste material.

Yet another object of this invention is to propose a formulation for the treatment of diabetes, which is simple to prepare.

These and other objects of the invention will be apparent to a reader on reading, the ensuing description.

### DETAILED DESCRIPTION OF THE INVENTION

Thus according to this invention is provided a formulation for the treatment of diabetes.

In accordance with this invention, a formulation comprising cow urine powder and skim milk powder, is prepared, for the treatment of diabetes.

The first early morning urine samples are collected from the cow shed. The cow urine samples are dried at temperatures such as ranging from 45° C. to 63° C. in a vacuum oven. Drying of cow urine samples at temperature 63° C. is found to be more effective than that of other temperature.

The fresh cow urine is filtered through a Whatman filter no 42 and the filtrate is heated at 45° C. to 63° C. in a vacuum oven up to 60 total soluble solids. The concentrate is filtered through Whatman filter No. 42 and the residue thus obtained is dried in a vacuum oven at 50° C. till dry. The dried material is kept in dessicators for further use. This powder obtained from cow urine does not carry the usual smell associated with it, and can thus be used freely without the normal repulsive feeling.

The process has been outlined in the Schematic diagram below (Scheme-1)

Chemical Analysis of Cow Urine Powder

The chemical analysis of cow urine powder includes the determination of total soluble solids (TSS), pH, moisture, calcium, potassium, nitrogen, urea, sulphate and creatinine.

Skim milk powder, is obtained commercially and is blended with the cow urine powder. The proportions in which cow urine powder and skim milk powder are mixed, for a given dose, are based on the following calculation:

I Dose: Human dose 1.24 g/day×(0.018×5) factor
a. =0.1116 g/kg body weight
b. =111.6=(112 mg/kg body weight)

II Dose: 2 (I dose)
a. =2×112
b. =224 mg/kg body weight

III Dose: Half of I dose
a. =56 mg/kg body weight
   Simultaneously dose of skim milk powder added to the cow urine powder and details are given in Table 1.

TABLE 1
Dose of the antidiabetic activity of the cow urine powder and SMP

| Cow Urine Powder (mg/kg) | Skim Milk Powder (mg/kg) | Total (mg/kg) |
|---|---|---|
| body weight | body weight | body weight |
| 56 mg | 44 mg | 100 mg |
| 112 mg | 88 mg | 200 mg |
| 224 mg | 176 mg | 400 mg |

One dose of skim milk powder was fixed 50 mg per rats that is skim milk powder control group.

The effect of the formulation has been studied on rats.

Selection and Registration

Albino Wistar rats of both sex (male and female) were procured from the laboratory of Columbia Institute of Pharmacy Tekari, Raipur 493111 (C.G.), India, and were maintained under a 12 h light/dark cycle and allowed for feed and water without limit. Approved experimental protocol in accordance with the guidelines provided by the committee for the control and supervision of experiments on animals (CPCSEA). The experimental rats were registered by CPCSEA and registration number was 1321/ac/10/CPCSEA.

Animal Details

A. Species: Wistar Albino rat

B. Age/weight: Adult, 80-250 gms

C. Gender: Either sex

D. Number to be use: 42 Rats

E. Proposed source of animals: Laboratory of Columbia Institute of Pharmacy

### 1. Tekari, Raipur 493111 (C.G.), India.

### Feeding Schedule

TABLE 2
Feeding and treatment schedule after induction
of diabetes in experimental animals

| | Quantity | | |
|---|---|---|---|
| Group | Treatment | Feeding without limit | Duration |
| Group I | Without treatment | Nutria and cheek pea | 8 hours |

Streptozotocin treated to all experimental animals
according to body weight (50 mg/kg body weight)

| | | | |
|---|---|---|---|
| Group II | Without treatment | Nutria and cheek pea | 8 hours |
| Group III | 1.44 mg/200 g/rats | Glyclazide | 24 hours |
| | | Nutria and cheek pea | 1 hours after |
| | | | treatment |
| Group IV | 50 mg/rats | Skim milk powder | 24 hours |
| | | Nutria and cheek pea | 1 hours after |
| | | | treatment |
| Group V | 100 mg/rats | Cow urine powder | 24 hours |
| | | SMP blends | |
| | | Nutria and cheek pea | 1 hours after |
| | | | treatment |
| Group VI | 200 mg/rats | Cow urine powder | 24 hours |
| | | SMP blends | |
| | | Nutria and cheek pea | 1 hours after |
| | | | treatment |
| Group VII | 400 mg/rats | Cow urine powder | 24 hours |
| | | SMP blends | |
| | | Nutria and cheek pea | 1 hours after |
| | | | treatment |

Note:
- Amounts/rats nutria 8 g and cheek pea 12 g and repeated after 8 hours.

During the experiments the blood sample was taken before treatment.

### Design of Experiment

Experiment design is given in table-3

TABLE 3
Design of experiment

| Group | No. of | |
|---|---|---|
| No. | animals | Treatment |
| 1 | 6 | Normal control (without diabetes) |
| 2 | 6 | Diabetic control (diabetes induced by |
| | | Streptozotocin, 50 mg/kg body weight/rats) |
| 3 | 6 | Standard drug group (1.44 mg/200 g rats/day) |
| 4/(a) | 6 | Skim milk powder (50 mg/rats/day) |
| 4/(b) | 6 | Cow urine powder SMP blends (100 mg/rats/day) |
| 4/(c) | 6 | Cow urine powder SMP blends (200 mg/rats/day) |
| 4/(d) | 6 | Cow urine powder SMP blends (400 mg/rats/day) |

Preparation of Streptozotocin Solution for Induction of Diabetes
a. M citrate buffer solution (pH 4.5)

An accurate quantity of 14.9 g of trisodium citrate was dissolved in 50-100 ml of volume distilled water made up to 1000 ml and pH of the buffer was adjusted to 4.5 by using conc. HCl.

Streptozotocin Solution

This solution was prepared by dissolving the weighed quantity of streptozotocin in above 0.1 M freshly prepared ice-cooled citrate buffer (pH 4.5) (Gururaja et al. 2011).

Induction of Diabetes in Different Groups of Rats Using Streptozotocin Solution

Diabetes was induced in rats by the intra peritoneal injection of streptozotocin (Himedia) at a dose of 50 mg/kg body weight dissolved in citrate buffer (0.1 M, pH 4.5) in the volume of 1 ml/kg. The diabetic rats were given 5% w/v glucose solution orally in order to prevent hypoglycemia during the first day after the streptozotocin administration. Three days after the injection (six group), the blood glucose levels were measured and the animals with blood glucose levels above 300 mg/dl were considered as being diabetic and were used in the subsequent experiments. In all the experiments, rats were fasted for 16 hr prior to streptozotocin injection.
a. Total units=202.5
b. 1 ml=40 units, 0.1 ml=4 units
c. Therefore total units 202.5/40 units=5.06 ml of streptozotocin solution
d. Freshly prepared streptozotocin solution (5.06 ml of solution/36 rats according to body weight) fed as a single dose only once to induce diabetes.

Treatment of Experimental Rats

Experimental rats were divided in 4 groups of 6 rats per group and 4^{th} group further divided in 4 groups with 6 rats in each sub group. The details of treatments were given below:

a. Group 1—Normal control group (without diabetes).

b. Group 2—Diabetic control group (streptozotocin-treated).

c. Group 3—Standard drug group—Diabetic animals received daily a single oral dose of the reference drug Glyclazide 1.44 mg/200 g body weight from day 1 to 15.

d. Group 4(a)—Diabetic animals received daily a single oral dose of SMP
i. 50 mg/rats from day 1 to 15.

e. Group 4(b)—Diabetic animals received daily a single oral dose of cow urine
i. powder SMP blends (100 mg).

f. Group 4(c)—Diabetic animal received daily a single oral dose of cow urine
i. powder SMP blends (200 mg).

g. Group 4(d)—Diabetic animals received daily a single oral dose of cow urine
i. powder SMP blends (400 mg).

Blood Sampling of Experimental Rats

Blood samples were collected retro-orbitally from the inner canthus of the eye under light ether anesthesia using capillary tubes (Micro Hemocrit Capillariea, Mucaps) as described by Sorg and Buckner (1964); Blood was transferred into fresh vials and serum was separated by centrifuging at 2000 rpm for 2 min. Blood glucose levels were measured using Accu-Check glucose kit.

Pharmacological Evaluation of Blood Sample

To observe the effect of administration of cow urine powder SMP blends and standard drug glyclazide diabetic rats was determined by measuring the fasting blood glucose levels and serum lipid profiles.

Day 3 of induction was designed as day 1 for administration of the standard drug glyclazide and cow urine SMP blends to diabetic rats, fasting blood glucose levels measured on days 1, 5, 10 and 15 of the experiment period. Other parameters were determined on after day 15 and residual study was carried out for another 15 days to observe the reappearance diabetes.

Glucose

Blood glucose was measured by using Accu-Check Active Glucometer (Roche) and the result was expressed as milligram per deciliter (mg/dl). This instrument is working on the principle of photometry.

Testing Time

With a testing time of about 5 seconds, Accu-Check Active is one of the fast blood glucose monitors.

Amount of Blood Sample

Accu-Check Active requires only a tiny drop of blood. One to two μl of blood drop was placed on the middle of the test pad. The reading directly read on the screen of the Accu-Check Active Glucometer.

Observation of Reaction

Application of blood to the test strip starts a color reaction. The final color was accurately read (measured photo metrically) by the meter's optical system and the result is converted to a blood glucose value (mg/dl). (WWW.accu-check.in)

Serum Lipid Profiles

The serum lipid profile parameters such as cholesterol, HDL, LDL and triglycerides content were studied by using blood serum obtained during the experiments.

Cholesterol

Cholesterol content in the blood samples were analyzed by using Bio Chemistry

Analyzer which is working on the principle of Lambert's-Beer's law.

For estimation of triglycerides, Bio-chemistry Analyzer was used.

Effect of cow urine powder SMP blends on blood glucose, cholesterol, HDL, LDL and triglyceride level in streptozotocin induced rats was tested and after 15 days of experiment the overall effect of the treatment in various control measures such as standard drug glyclazide, skim milk powder and various levels of cow urine powder SMP blends (100, 200 and 400 mg) was observed.

Results obtained during the treatment period for blood glucose level in various groups such as normal control, diabetic control, standard drug glyclazide, skim milk powder, cow urine powder SMP blend 100 mg, cow urine powder SMP blend 200 mg and cow urine powder SMP blend 400 mg were 91.66, 447.66, 125.25, 222.75, 174.75, 163.25, and 133.50 mg/dl respectively. The lipid profile levels were determined during the experiment for all groups. In normal control group, diabetic group, standard drug group, skim milk powder group and cow urine powder SMP blends group (100, 200 and 400 mg) the cholesterol level was 81.65, 126.37, 67.72, 117.42, 95.55, 93.70, and 90.82 mg/dl respectively. The value obtained for LDL level for various groups such as normal control group, diabetic control group, standard drug group, skim milk powder group, cow urine powder SMP blend group 100 mg, cow urine powder SMP blend group 200 mg and cow urine powder SMP blend group 400 mg were 28.11, 86.87, 16.17, 76.12, 51.15, 47.22, and 40.85 mg/dl respectively. Similarly the HDL level for various groups such as normal control group, diabetic control group, standard drug group, skim milk powder group, cow urine powder SMP blend group 100 mg, cow urine powder SMP blend group 200 mg and cow urine powder SMP blend group 400 mg were 53.53, 39.50, 51.55, 41.30, 44.40, 46.47, and 49.97 mg/dl respectively. The samples were also analyzed for triglyceride content in the normal control group, diabetic control group, standard drug group, skim milk powder group, cow urine powder SMP blend group 100 mg, cow urine powder SMP blend group 200 mg, and cow urine powder SMP blend group 400 mg and the triglyceride contents were 77.21, 188.67, 95.35, 128.72, 134.55, 132.70 and 126.47 mg/dl respectively.

The table reveals that glucose level for normal control group was 91.6 mg/dl and streptozotocin was introduced in normal group and rats become diabetic and blood glucose level was 447.6 mg/dl. After induction of diabetes in the rats they were treated with standard drug glyclazide and the blood glucose level was reduced from 599.1 to 125.2 mg/dl., within 15 days of experiment. Similarly in the other groups such as skim milk powder and the various level of cow urine powder SMP blends 100, 200 and 400 mg the blood glucose level reduced significantly (p<0.01) from 494.0 to 222.7, from 632.0 to 174.7, from 547.6 to 164.6 and from 630.5 to 133.6 mg/dl respectively.

The cholesterol level for normal control group was 81.65 mg/dl whereas in diabetic group the blood cholesterol level was 126.37 mg/dl. After induction of diabetes in the rats, they were treated with standard drug glyclazide and it was observed that the blood cholesterol level was reduced from 125.44 to 67.72 mg/.dl. It was also observed that in the skim milk powder group and the various levels of cow urine powder SMP blends group (100, 200 and 400 mg), the blood cholesterol level reduced significantly (p<0.01) from 132.63 to 117.42, from 134.23 to 95.55, from 132.88 to 93.70 and from 133.71 to 90.82 mg/dl respectively. On the other hand, LDL level for normal control group was 28.11 mg/dl and in diabetic group the blood LDL level was 86.87 mg/dl and in glyclazide group the blood LDL level was reduced from 83.73 to 16.17 mg/dl. Whereas in skim milk powder group and the various levels of cow urine powder SMP blends groups (100, 200 and 400 mg), the blood LDL reduced significantly (p<0.01) from 96.38 to 76.12, from 99.35 to 51.15, from 98.43 to 47.22 and from 98.53 to 40.85 mg/dl respectively. HDL level estimated for normal control group was 53.53 mg/dl and in diabetic group the blood HDL level was 39.50 mg/dl. In glyclazide group the blood HDL level was increased from 41.75 to 51.55 mg/dl. Whereas skim milk powder group and cow urine powder SMP blends groups (100, 200 and 400 mg) the blood HDL level increased significantly (p<0.01) from 36.39 to 41.30, from 34.90 to 44.40, from 34.42 to 46.47 and from 35.25 to 49.97 mg/dl respectively. Triglyceride level also measured for normal control group was 77.21 mg/dl and in diabetic group the blood triglyceride level was 188.67 mg/dl. Standard drug glyclazide group content the triglyceride level and it was reduced from 187.96 to 95.35 mg/dl. Other groups such as skim milk powder group and the various levels of cow urine SMP powder blends groups (100, 200 and 400 mg) the triglyceride level reduced significantly (p<0.01) from 167.15 to 128.72, from 189.36 to 134.55, from 179.73 to 132.70 and from 188.56 to 126.47 mg/dl respectively.

Data obtained during the experimental for standard drug group, skim milk powder group and cow urine powder SMP blends groups (100, 200 and 400 mg) were significantly higher than the studies conducted by Gururaja et al. (2011) on antidiabetic potential of cow urine distillate and found significant reduction of the elevated blood glucose, serum cholesterol and serum triglyceride levels when compared with the diabetic control. The diabetic animals treated with cow urine distillate also showed a significant increase in HDL levels when compared with the diabetic controls.

In present investigation it was also observed that the cow urine powder SMP blends (400 mg) significantly reduced the blood glucose level and lipid profile level than that of 100 mg and 200 mg. This may be due to presence of antioxidant and free radical scavengers in cow urine powder. This might be responsible for the observed antidiabetic and lipid profile reduction effect in the experimental animals.

TABLE 4
Effect of Cow urine powder SMP blends on blood glucose and lipid
profile in streptozotocin treated diabetic rats after 15th day

| | Blood | Blood | | | | |
|---|---|---|---|---|---|---|
| | glucose on | glucose on | Lipid profile (mg/dl) | | | |
| Groups | 1^{st} day | 15^{th} day | Cholesterol | LDL | HDL | Triglyceride |
| Normal control (T₁) | 92.33 ± 6.8 | 91.66 ± 1.70 | 81.65 ± 2.77 | 28.11 ± 2.23 | 53.53 ± 1.42 | 77.21 ± 2.49 |
| Diabetic control (T₂) | 357.80 ± 90.03 | 447.66 ± 32.69 | 126.37 ± 6.07 | 86.87 ± 7.88 | 39.50 ± 2.93 | 188.67 ± 16.6 |
| Standard Glyclazide | 599.16 ± 57.10 | 125.25 ± 12.33 | 67.72 ± 3.37 | 16.17 ± 5.25 | 51.55 ± 1.91 | 95.35 ± 3.10 |
| 1.44 mg (T₃) | | (79.09%) | | | | |
| SMP | 494.00 ± 63.87 | 222.75 ± 27.12 | 117.42 ± 4.63 | 76.12 ± 4.43 | 41.30 ± 2.11 | 128.72 ± 1.74 |
| 50 mg/animal/day (T₄) | | (54.82%) | | | | |
| Cow urine SMP blends | 632.00 ± 27.45 | 174.75 ± 17.10 | 95.55 ± 3.09 | 51.15 ± 0.86 | 44.40 ± 2.93 | 134.55 ± 4.51 |
| 100 mg/animal/day (T₅) | | (72.34%) | | | | |
| Cow urine SMP blends | 547.60 ± 77.97 | 163.25 ± 28.44 | 93.70 ± 3.06 | 47.22 ± 2.87 | 46.47 ± 3.85 | 132.70 ± 5.61 |
| 200 mg/animal/day (T₆) | | (70.18%) | | | | |
| Cow urine SMP blends | 630.50 ± 51.47 | 133.50 ± 6.61** | 90.82 ± 1.93 | 40.85 ± 4.94 | 49.97 ± 3.52 | 126.47 ± 6.02 |
| 400 mg/animal/day (T₇) | | (78.82%) | | | | |

| Group | CD | CV | F. Cal | SE± |
|---|---|---|---|---|
| T1-T2 | 74.69 | 21.02 | 34.51 | 06.61 |
| T2-T3 | 80.67 | | | |
| T3-T4 | 74.69 | | | |
| T3-T5 | 74.69 | | | |
| T3-T6 | 74.69 | | | |
| T3-T7 | 74.69** | | | |

Values are the average of 6 (six) replication (six animal)
**At 1% level of significance, % percent reduce

Effect on Glucose Levels of Diabetic Rat Treated with Various Control Measures During the Residual Period

Residual effect of standard drug glyclazide group, skim milk powder group and cow urine powder SMP blends groups (100, 200 and 400 mg) for residual of blood glucose levels in streptozotocin treated rats for 15 days and observations were recorded.

Data given in table 4 indicated that the residual effect on glucose levels in diabetic rats treated with standard drug glyclazide, skim milk powder and various levels of cow urine powder SMP blends (100, 200, 400 mg) during the 15 days of residual periods.

Results obtained during residual period on 20th, 25th and 30th days blood glucose level in various group such as normal control group, standard drug glyclazide group, skim milk powder group and various levels of cow urine powder SMP blends groups (100, 200, 400 mg) were 91.61, 483.36, 176.50, 214.75, 173.23, 150.53, 134.56 and 90.84, 532.33, 174.76, 223.07, 174.29, 163.55, 134.70 and 91.04, 561.53, 201.57, 222.74, 169.02, 161.78, 135.04 mg/dl respectively.

The table reveals that during residual period glucose level for normal control group was nearly stable. Whereas streptozotocin was introduced in normal group to make it diabetic then the blood glucose level raised for 483.36 to 561.53 mg/dl. after withdrawal of treatment with standard drug glyclazide. It was observed that the blood glucose level was raised to 176.50 to 201.57 mg/dl. During residual period in other skim milk powder and various levels of cow urine powder SMP blends (100, 200, 400 mg) treated groups were in steady state.

TABLE 5
Effect on glucose levels of diabetic rat treated with
various control measures during the residual period

| | Dose | Blood glucose level | | |
|---|---|---|---|---|
| Groups | (mg/kg) | 20^{th} Day | 25^{th} Day | 30^{th} Day |
| Normal | | 91.61 ± 1.7 | 90.84 ± 2.02 | 91.04 ± 1.65 |
| control | | | | |
| (T₁) | | | | |
| Diabetic | | 483.36 ± 28.82 | 532.33 ± 24.06 | 561.53 ± 38.50 |
| control | | | | |
| (T₂) | | | | |
| Standard | 1.44 | 176.50 ± 29.75 | 174.76 ± 27.42 | 201.57 ± 29.85 |
| Glyclazide | (mg/ | | | |
| (T₃) | 200 g) | | | |
| SMP (T₄) | 50 mg/ | 214.75 ± 17.78 | 223.07 ± 21.82 | 222.74 ± 24.87 |
| | animal/ | | | |
| | day | | | |
| Cow urine | 100 mg/ | 173.27 ± 14.68 | 174.29 ± 14.22 | 169.02 ± 12.21 |
| SMP | animal/ | | | |
| blends | day | | | |
| (T₅) | | | | |
| Cow urine | 200 mg/ | 150.53 ± 20.65 | 163.55 ± 24.57 | 161.78 ± 27.21 |
| SMP | animal/ | | | |
| blends | day | | | |
| (T₆) | | | | |
| Cow urine | 400 mg/ | 134.56 ± 5.90 | 134.70 ± 5.10 | 135.04 ± 6.05** |
| SMP | animal/ | | | |
| blends | day | | | |
| (T₇) | | | | |

| | CD | | |
|---|---|---|---|
| | 20^{th} Day blood | 25^{th} Day blood | 30^{th} Day blood |
| Group | glucose | glucose | glucose |
| T₁-T₂ | 70.62 | 71.52 | 90.63 |
| T₂-T₃ | 76.28 | 77.25 | 96.13 |
| T₃-T₄ | 70.62 | 71.52 | 78.49 |
| T₃-T₅ | 70.62 | 71.52 | 78.49 |
| T₃-T₆ | 70.62 | 71.52 | 78.49 |
| T₃-T₇ | 70.62 | 71.52 | 78.49 |
| CV | 19.04 | 18.51 | 21.00 |
| F. Cal | 44.95 | 55.85 | 38.63 |

Values are the average of 6 (six) replication (six animal)
**At 1% level of significance

Encapsulation of Standardized Dose

For the encapsulation of standardized dose of cow urine powder SMP blends i.e. 400 mg, cellulose capsules were used. The material of capsule is of Hydroxypropymethyl cellulose (HPMC) i.e. the mixture of 92% of methyl cellulose and 8% of purified water.

The details of filling capacities on the basis of capsule size are as below:—

a. 1. Cow urine powder SMP blends density:—08 g/ml
b. 2. Capsule size:—1
c. 3. Filling capacity in mg:—400 mg
For the filling of capsules, blending ratio of cow urine powder and SMP were maintained as 224:176 (400 mg). The final composition of effective dose of capsule is given below:—

TABLE 6
Proximate composition per capsule

| | Particulars of | | | |
|---|---|---|---|---|
| S. No. | constituents | Contents/100 g | | Content/400 mg |
| 1. | Total nitrogen (N₃) | 1.79 | g | 7.16 mg |
| 2. | Urea (CH₄N₂O) | 21.44 | g | 87.76 mg |
| 3. | Calcium (Ca) | 113.39 | mg | 0.45 mg |
| 4. | Potassium (K) | 164.27 | mg | 0.65 mg |
| 5. | Sulphate (SO₄) | 83.48 | mg | 0.33 mg |
| 6. | Creatinine | 154.12 | mg | 0.61 mg |
| | (C₆HgN₂O₂) | | | |

Final packaging of the capsule can be done in aluminum foil and can be stored at ambient temperature.

### Capsule Dimension and Filling Capacities

Table 7 shows the capsule sizes with their respective filling capacities.

TABLE 7
Example for capsule dimensions and filling capacities

| | | |
|---|---|---|
| Capsule | Capsule | Capacity in mg powder density |

| size | volume in ml | 0.6 | 0.8 | 1.0 | 1.2 g/ml |
|---|---|---|---|---|---|
| 000 | 1.37 | 822 | 1096 | 1370 | 1644 |
| 00el | 1.02 | 612 | 816 | 1020 | 1224 |
| 00 | 0.91 | 546 | 728 | 910 | 1092 |
| 0el | 0.78 | 468 | 624 | 780 | 936 |
| 0 | 0.68 | 408 | 544 | 680 | 816 |
| 1 | 0.50 | 300 | 400 | 500 | 600 |
| 2 | 0.37 | 222 | 296 | 370 | 444 |
| 3 | 0.30 | 180 | 240 | 300 | 360 |
| 4 | 0.21 | 126 | 168 | 210 | 252 |
| 5 | 0.10 | 78 | 104 | 130 | 156 |

What is claimed is:

1. A method of treating diabetes, comprising administering to a patient in need of treatment for diabetes, a formulation comprising cow urine powder and skim milk powder in an amount effective to treat symptoms of diabetes.

2. The method of claim 1, wherein the formulation comprises between 56 and 224 mg/kg of cow urine powder and between 44 and 176 mg/kg of skim milk powder.

3. The method of claim 2, wherein the amount of formulation administered to the patient is between 100 and 400 mg/kg.

4. The method of claim 1, wherein the symptoms are one or more of hyperglycemia, high cholesterol, and high triglycerides.

5. The method of claim 1, wherein the formulation is administered once daily.

6. A method of treating diabetes, comprising administering to a patient in need of treatment for diabetes, a formulation consisting of cow urine powder and skim milk powder in an amount effective to treat symptoms of diabetes.

7. The method of claim 6, wherein the formulation comprises between 56 and 224 mg/kg of cow urine powder and between 44 and 176 mg/kg of skim milk powder.

8. The method of claim 7, wherein the amount of formulation administered to the patient is between 100 and 400 mg/kg.

9. The method of claim 6, wherein the symptoms are one or more of hyperglycemia, high cholesterol, and high triglycerides.

10. The method of claim 6, wherein the formulation is administered once daily.
